(19) United States
(12) Patent Application Publication
(10) Pub. No.: US 2010/0076518 A1
(43) Pub. Date: Mar. 25, 2010

(54) SYSTEMS AND METHODS FOR RELIEVING DYSPNEA

(75) Inventors: Edwin J. Hlavka, Minneapolis, MN (US); Lynn S. Elliott, Maple Grove, MN (US); Joyce Wahr, Minnetonka, MN (US)

Correspondence Address: PERKINS COIE LLP;PATENT-SEA, P.O. BOX 1247, SEATTLE, WA, 98111-1247, US

(73) Assignee: ConcepTx Medical, Inc., Minneapolis, MN (US)

(21) Appl. No.: 12/561,918

(22) Filed: Sep. 17, 2009

Related U.S. Application Data
(60) Provisional application No. 61/098,198, filed on Sep. 18, 2008.

(51) Int. Cl. A61N 1/36

(52) U.S. Cl. 607/42

(57) ABSTRACT

The present disclosure is directed generally to systems and methods for relieving dyspnea. A method in accordance with a particular embodiment includes receiving an input signal from a patient sensor, the input signal corresponding to an indication of the patient's breathing. The method can further include, based at least in part on the input signal, at least reducing the patient's sensation of dyspnea by delivering electrical stimulation to at least one electrode, the at least one electrode being positioned in signal communication with at least one of the patient's inspiratory muscles, expiratory muscles, afferent neural pathways of the inspiratory muscles, and afferent neural pathways of the expiratory muscles.

### CROSS-REFERENCE TO RELATED APPLICATION

The present application claims priority to U.S. Provisional Application No. 61/098,198, filed Sep. 18, 2008 and incorporated herein by reference.

### TECHNICAL FIELD

The present disclosure is directed to systems and methods for relieving dyspnea, including via electrical signals delivered to breathing muscles and/or associated afferent neural pathways.

### BACKGROUND

Dyspnea is the chief patient complaint in a variety of diseases of the pulmonary system. These diseases include chronic bronchitis (12.5 million US patients), emphysema (1.7 million US patients), and asthma (18 million US patients), collectively referred to as Chronic Obstructive Pulmonary Diseases, or COPD. Dyspnea is also reported by patients suffering from combinations of the foregoing diseases, and/or other pulmonary diseases, and non-pulmonary diseases (notably in heart failure). Dyspnea, while a common medical term, is actually poorly defined and ultimately subjective since it is generally the perception of difficulty breathing or difficulty catching one's breath, and more generally, an uncomfortable sensation of breathing.

The severity of pulmonary diseases can typically be measured using objective techniques, such as FEV1 (the patient's forced expiratory volume in the first second of exhalation), minute ventilation (the volume inhaled or exhaled by the patient in one minute), arterial blood gas levels (e.g., of oxygen or carbon dioxide), among others. By contrast, the patient's dyspnea experience can be simply one of difficulty breathing, ultimately leading to a reduction or elimination of physical activity due to this discomfort. That is, the patient complaint is of dyspnea and a loss of mobility or physical function, not of a decreased FEV1.

In many ways dyspnea can be analogous to the perception of pain. While an organic source of the pain may be present (a broken bone, for example), the pain itself can be a problem and may require palliative treatment. Furthermore, in the same way that an individual can suffer from chronic pain for which an organic cause is either absent or inadequate to cause the pain, some patients can suffer from severe dyspnea despite relatively normal objective measures of pulmonary performance.

The origins of dyspnea remain unclear. Studies and experience have yielded confusing and often seemingly contradictory results. Treatments for dyspnea range from supplemental oxygen therapy to sitting in front of a fan to systemic opiates. Furthermore, dyspnea can be experimentally induced by vigorous exercise, breath-holding, breathing through a restrictive mouthpiece, or breathing carbon dioxide in symptomatic pulmonary disease patients. A common, though unproven, theory is that dyspnea derives from a mismatch between outgoing motor signals to the respiratory muscles and incoming afferent information. In one example, under a give set of conditions, the brain can expect a certain pattern of ventilation and associated afferent feedback. Deviations from this pattern can cause or intensify the sensation of dyspnea.

While dyspnea is often the chief complaint of a patient, there is currently no pharmacologic agent that primarily treats dyspnea. That is, a variety of bronchodilators are used to treat asthma and other COPD, and while they demonstrably increase FEV1, their effects on dyspnea can be modest and can fall below that of clinical significance. Accordingly, there remains a need for methods and devices that effectively treat dyspnea.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

Table 1 is a table of various categories and therapeutic methods that can be used to affect the sensation of dyspnea.

FIG. 1 illustrates a variety of inputs and control loops that can contribute to the sensation of dyspnea.

FIG. 2 shows an electrical stimulator according to one example of the present subject matter.

FIG. 3 shows muscle and muscle locations related to inspiration and expiration, based on plate 183 of “Atlas of Human Anatomy,” 2nd Edition, by Frank Netter (Icon Learning Systems 2001), hereinafter “Netter.”

FIG. 4 shows intercostal nerves and arteries of a patient, based on plate 179 of Netter.

FIG. 5 shows a right lateral view of posterior intercostal veins and arteries, based on plate 218 of Netter.

FIG. 6 shows a signal generator implanted in a subclavicular region according to one example of the present subject matter.

FIG. 7 shows a signal generator implanted in a renal region according to one example of the present subject matter.

### DETAILED DESCRIPTION

### Chest Wall Vibration

Mechanical vibration of the chest wall can relieve the sensation of dyspnea. Mechanical “in phase” vibration (i.e., vibration applied to the contracting muscles, the inspiratory intercostal muscles during the inspiratory phase of breathing) reduces the sensation of dyspnea. “Out of phase” vibrations often have a detrimental effect, increasing the sensation of dyspnea.

Dyspnea can be relieved using mechanical vibrators applied to the external chest wall in an upper anterior (typically the 2^{nd} or 3^{rd} intercostal spaces) parasternal location. In one example, manual application of the vibrators during inspiration and not during exhalation reduces dyspnea. In some examples, semi-permanently applied vibrators with sensors and a control algorithm trigger the vibrators during the inspiratory phase. An example of semi-permanent application of the vibrators includes placing the vibrators in a vest worn by a patient. An example of a sensor for detecting the inspiratory phase of a patient includes a thermistor placed at or in the patient's nose to sense airflow associated with the patient's breath. It is understood that other applications of the vibrators and selection of sensors is possible without departing from the scope of the present subject matter.

Mechanical vibrators used for chest wall vibration are typically 25 mm in diameter with an amplitude of 2 mm at a 100-120 Hz frequency. Such a transducer is bulky, noisy, and consumes substantial energy. Thus, a practical solution utilizing such devices would also be bulky, requiring a vest or other means to clamp the device to the chest, an electrical power supply, a separate sensor to detect airflow (likely placed near the nose or mouth), and a control device. Such a practical solution can be bulky, heavy, inconvenient, and unsightly for the user and would not be highly desired.

The mechanical vibration functionally serves to stimulate the intercostal muscle fibers to provide additional afferent feedback from the skeletal muscles to “trick” the body into sensing that it is breathing more and easier. In other embodiments, other mechanisms provide additional afferent feedback from the intercostal muscles to provide the same benefit.

In one example, electrical stimulation of the intercostal muscles is used to produce contractions (i.e., TENS transcutaneous electrical nerve stimulation). Electrical stimulators are of lower bulk and power usage than mechanical vibrators. In various examples, a single chest “strap” (similar in outward design to an athletic pulse monitor) can be used to place and hold the electrodes as well as to sense chest wall expansion during inspiration. A control unit provides control of the TENS electrodes based upon sensing inspiration.

A battery pack to power the device can be located in a variety of places, including but not limited to, on the strap, remotely, such as on a belt holster, via a connecting power cord or a combination thereof.

In another example, electrical stimulation of the afferent nerves leading from the intercostal muscles can duplicate the effects (on the central sensory cortex) of mechanical vibration of those same intercostal muscles. Electrical stimulation of the afferent nerves leading from the intercostal muscles can use less electrical power than either mechanical vibration or TENS. In various examples, implanted electrodes are located to effectively inject appropriate electrical signals into the afferent nervous fibers. One example includes a fully implantable system where the electrodes, leads, signal generator and battery, and sensors are all permanently implanted under the skin.

In various examples, the sensors for identifying the inspiratory phase of breathing include, but are not limited to, an external chest strap (such as described above) communicating transcutaneously with the signal generator, a strain gage implanted on the chest wall to measure chest expansion directly, a temperature-based flow meter (thermistor or thermocouple) implanted in the mucosa of the respiratory tract to sense airflow, impedance plethysmography utilizing permanently implanted leads, including both electrodes and sensor placed on the chest wall, or sensing directly, either through mechanical or electrical means, the firing of the intercostal muscles.

The intercostal nerve carrying afferent signals from the intercostal muscles runs parallel to the intercostal space from the sternum back around to the sympathetic chain and then to the spinal cord. An electrical lead can be placed at a variety of locations ranging from anterior to posterior. The leads can be tunneled subcutaneously to a convenient location for the signal generator after one or more electrodes are placed on one or more intercostal nerves. Lead tunnel locations include, but are not limited to subclavicular, on the abdominal wall, and on the back over the kidney. Sensor leads (such as from impedance plethysmography) could be similarly tunneled.

Some examples include accommodations for percutaneous recharging of the battery and for programming/controlling of the signal generator.

While certain embodiments of this disclosure are directed to stimulating the inspiratory muscles, such as the external intercostals, during inspiration only (“in-phase” vibrations), in other embodiments, the expiratory muscles can be stimulated, in addition to or in lieu of stimulating the inspiratory muscles. In the case of the expiratory muscles, such as the internal intercostals and/or the abdominals, “in-phase” refers to stimulation during the expiratory phase of breathing only.

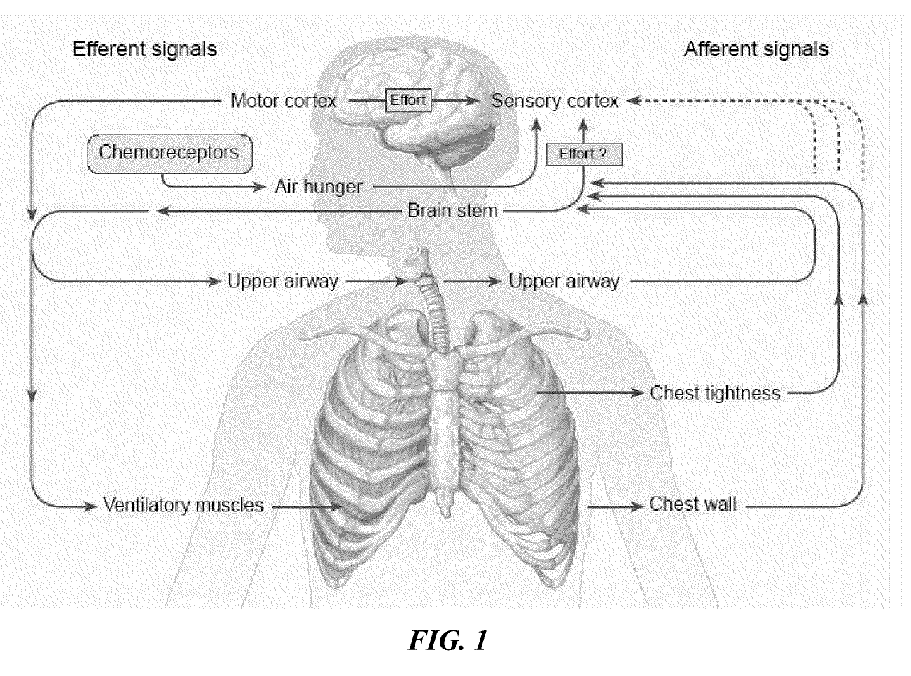

As illustrated in FIG. 1, a variety of inputs or control loops can contribute to the subjective sensation of dyspnea. Similarly, Table 1 generally illustrates various categories or therapeutic methods that can be used to affect the sensation of dyspnea.

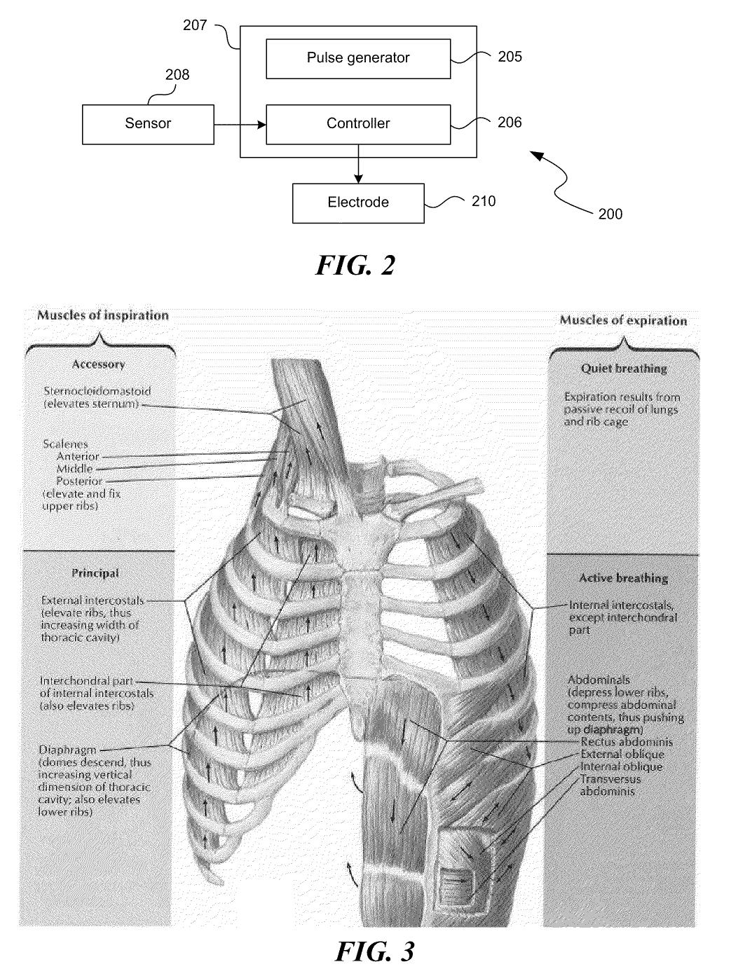

FIG. 2 shows an electrical stimulator system 200 according to one example of the present subject matter. The system 200 can include a housing 207 that contains a pulse generator 205 and a controller 206. The housing 207 can be an implantable housing, or it can be configured to be worn externally by the patient. When implanted, the housing 207 can be positioned at a subclavicular or other suitable location. The controller 206 can include a processor and memory, either or both of which include a computer-readable medium programmed with instructions for delivering electrical signals in automatic response to input signals. The input signals can be provided by one or more sensors 208 (e.g., thermistors, strain gages or other sensors) and the electrical signal can be delivered to one or more electrodes 210 (e.g., transcutaneous electrodes or implanted electrodes).

The electrical signal can be delivered to the electrodes 210 in accordance with selected stimulation parameters. For example, when the electrodes 210 are implanted and the signal is delivered to muscle tissue, it can be delivered at a current amplitude of up to 10 mA, a frequency of from about 0.1 Hz to about 50 Hz, and a pulsewidth of from about 0.05 milliseconds to about 5 milliseconds. The signal can be provided in bursts, timed to the patient's inspiration and/or expiration, lasting from about 0.1 seconds to about 5 seconds. When the signal is directed from an implanted electrode to neural tissue, the current amplitude can be up to about 10 mA, the frequency can range from about 1 Hz to about 100 Hz, with a pulsewidth of from about 25 microseconds to about 250 microseconds. Signal burst characteristics can be similar to those described above for muscle stimulation. Signals provided by an external electrode (e.g., placed on the patient's skin) can have a current amplitude of up to about 100 mA, a frequency of from about 0.1 Hz to about 150 Hz, a pulsewidth of from about 25 microseconds to about 1000 microseconds, and bursts having a timing and duration similar to those described above.

FIG. 3 shows muscle and muscle locations related to inspiration and expiration. One or more electrodes can be positioned to stimulate inspiratory muscles, expiratory muscles, or both. As discussed above, the stimulation is generally in phase with the activity of the inspiratory and/or expiratory muscles to facilitate the patient's sensation of comfortable (or less uncomfortable) breathing, thus reducing or eliminating the patient's sensation of dyspnea.

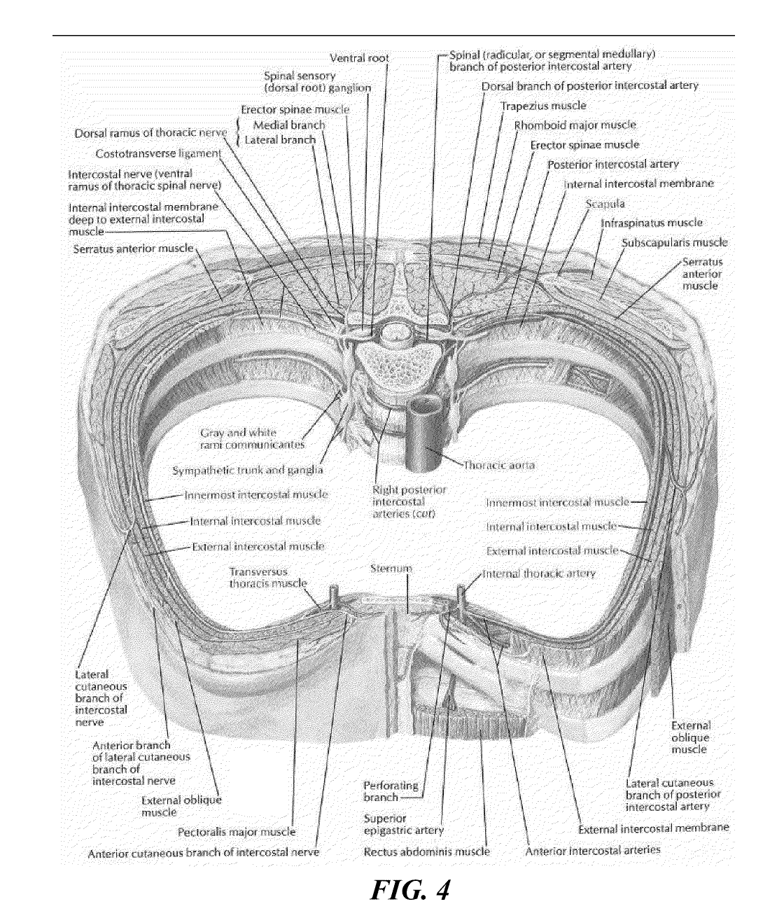

FIG. 4 shows intercostal nerves and arteries of a patient. In particular embodiments, the afferent nerves of the expiratory and/or inspiratory muscles can be stimulated in addition to or in lieu of stimulating the muscles themselves, to facilitate reducing or eliminating the patient's sensation of dyspnea.

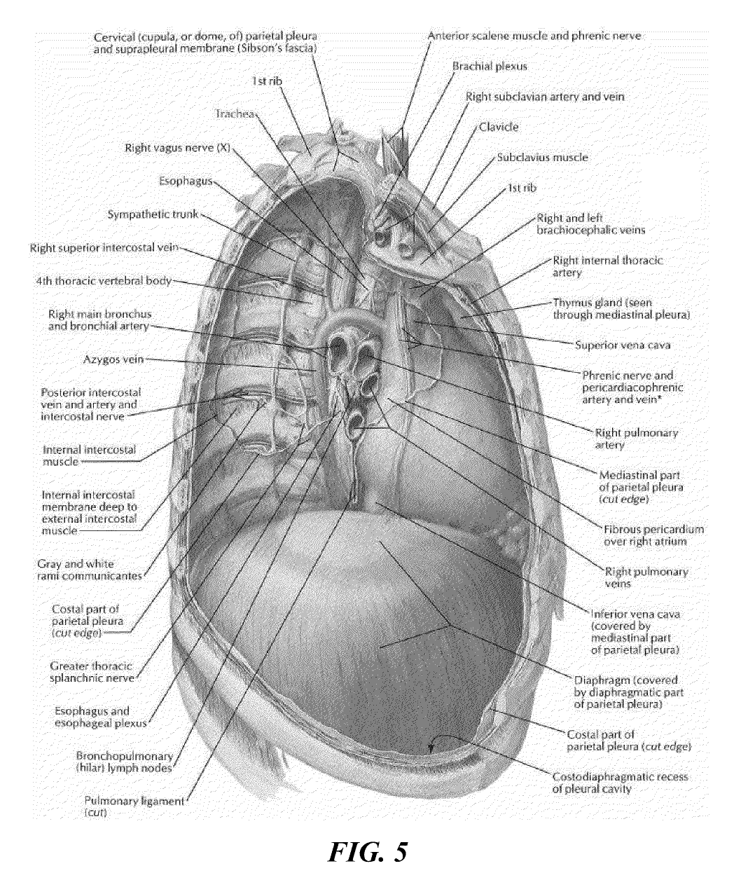

FIG. 5 shows a right lateral view of posterior intercostal veins and arteries.

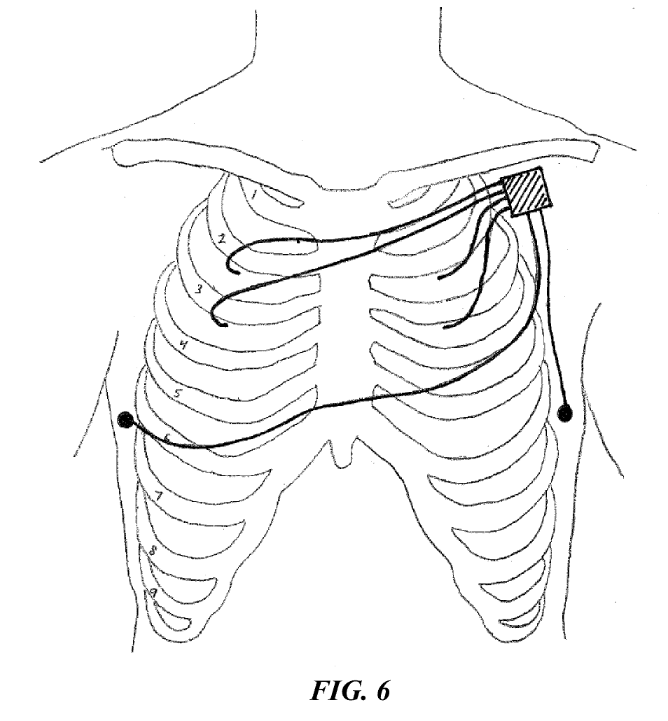

FIG. 6. shows a signal generator implanted in subclavicular region according to one example of the present subject matter. Four leads connect to bilateral parasternal electrodes at the second and third intercostal spaces. Sensors for impedance plethysmography are located at the lateral margins of the thoracic cavity.

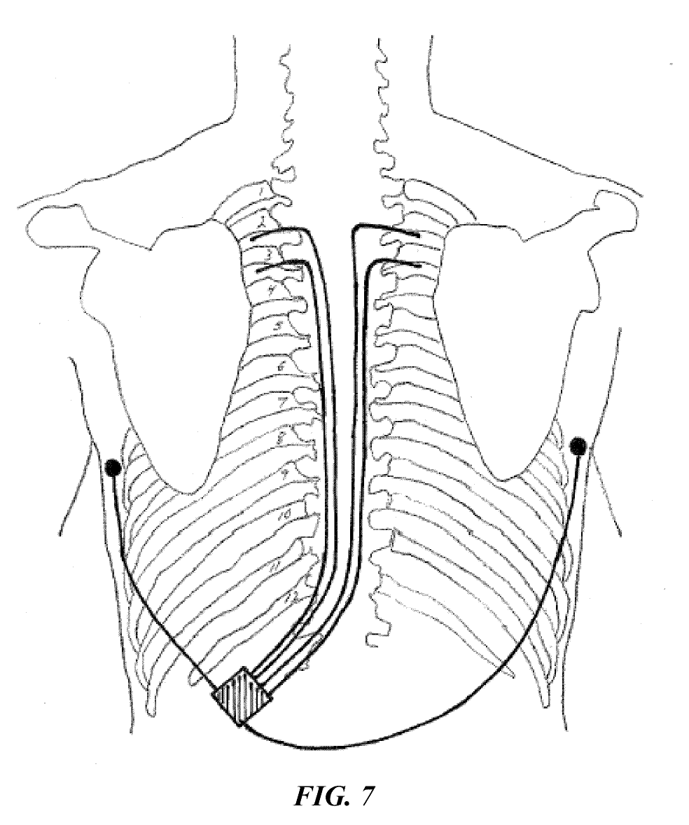

FIG. 7 shows a signal generator implanted in a renal region according to one example of the present subject matter. Four leads connect to bilateral paraspinal electrodes at the second and third intercostal spaces. Sensors for impedance plethysmography are located at the lateral margins of the thoracic cavity.

As discussed above, at least one advantage associated with one or more of the embodiments of the disclosed systems and methods is that they can provide the patient with relief from dyspnea. In particular embodiments that include electrical stimulation to achieve the foregoing results, the systems can be more effective, less bulky, and/or can consume less power than systems that mechanically stimulate the patient's muscles.

From the foregoing, it will be appreciated that specific embodiments of the subject technology have been described herein for purposes of illustration, but that various modifications may be made without deviating from the technology. For example, sensors other than those expressly disclosed above may be used to provide feedback to the controller. The electrodes used to provide stimulation to the patient can have any of a variety of configurations, including cuff electrodes for stimulating neural tissue, and disk-shaped electrodes for transcutaneously stimulating muscle tissue. Certain aspects of the disclosure described in the context of particular embodiments may be combined or eliminated in other embodiments. Further, while advantages associated with certain embodiments have been described in the context of those embodiments, other embodiments may also exhibit such advantages. Not all embodiments need necessarily exhibit such advantages to fall within the scope of the present technology. Accordingly, the present disclosure can include other embodiments not expressly shown or described above.

TABLE 1
THERAPEUTIC INTERVENTIONS AND THEIR TIE TO
PATHOPHYSIOLOGIC MECHANISM

| Pathophysiologic Mechanism | Therapeutic Intervention |
|---|---|
| Reduce ventilatory demand | |
| Reduce metabolic load | Exercise training: improve efficiency |
| | of CO₂ elimination |
| | Supplemental O₂ therapy |
| | Supplemental O₂ therapy |
| Decrease central drive | Pharmacologic therapy: |
| | Opiate therapy |
| | Anxiolytic therapy |
| | Alter pulmonary afferent information: |
| | Vibration |
| | Ventilator settings |
| | Inhaled pharmacologic therapy |
| | Fans |
| | Improve efficiency of CO₂ elimination: |
| | Altered breathing pattern |
| Reduce ventilatory impedance | |
| Reduce/counterbalance | Surgical volume reduction: |
| lung hyperinflation | Continuous positive airway pressure |
| Reduce resistive load | Pharmacologic therapy |
| Improve inspiratory | Nutrition |
| muscle function | Inspiratory muscle training |
| | Positioning |
| | Partial ventilatory support |
| | Minimizing use of steroids |
| Alter central perception | Education |
| | Cognitive-behavioral approaches |
| | Desensitization |
| | Pharmacologic therapy |

1. A method for treating a patient, comprising:
placing at least one electrode in signal communication with at least one of the patient's inspiratory muscles, expiratory muscles, afferent neural pathways of the inspiratory muscles, and afferent neural pathways of the expiratory muscles;
placing at least one sensor in a position to detect the patient's breathing;
coupling the at least one electrode and the at least one sensor to a controller; and
activating the controller to at least reduce the patient's sensation of dyspnea by delivering electrical stimulation to the at least one electrode based at least in part on input received from the at least one sensor.

2. The method of claim 1 wherein placing the at least one electrode includes implanting the at least one electrode.

3. The method of claim 1 wherein placing the at least one electrode includes attaching the at least one electrode to the patient's skin.

4. The method of claim 1 wherein placing the at least one electrode includes placing the at least one electrode to stimulate the patient's breathing muscles.

5. The method of claim 1 wherein activating the controller includes activating the controller to deliver electrical stimulation that is in phase with the patient's breathing.

6. The method of claim 5 wherein the electrical stimulation is delivered in phase with the patient's inspiratory activity.

7. The method of claim 5 wherein the electrical stimulation is delivered in phase with the patient's expiratory activity.

8. The method of claim 5 wherein the electrical stimulation is delivered in phase with the patient's inspiratory and expiratory activity.

9. The method of claim 1 wherein placing the at least one sensor includes placing a thermistor in the patient's breathing passage.

10. The method of claim 1 wherein placing the at least one sensor includes placing a strain sensor at the patient's chest.

11. The method of claim 1 wherein placing the at least one sensor includes implanting the at least one sensor.

12. The method of claim 1 wherein placing the at least one sensor includes attaching the sensor to the patient's skin.

13. The method of claim 1, further comprising implanting the controller.

14. The method of claim 1 wherein:
placing the at least one electrode includes implanting four bilateral parasternal electrodes at the patient's second and third intercostal spaces;
activating the controller includes activating the controller to deliver electrical stimulation in phase with the patient's breathing; and
placing the at least one sensor includes implanting a strain gage at the patient's chest wall; and wherein the method further comprises
implanting the controller at a subclavicular region of the patient.

15. A method for treating a patient, comprising:
receiving an input signal from a patient sensor, the input signal corresponding to an indication of the patient's breathing; and
based at least in part on the input signal, at least reducing the patient's sensation of dyspnea by delivering electrical stimulation to at least one electrode, the at least one electrode being positioned in signal communication with at least one of the patient's inspiratory muscles, expiratory muscles, afferent neural pathways of the inspiratory muscles, and afferent neural pathways of the expiratory muscles.

16. The method of claim 15 wherein delivering electrical stimulation includes delivering electrical stimulation that is in phase with the patient's breathing.

17. The method of claim 15 delivering electrical stimulation includes delivering electrical stimulation that is in phase with the patient's inspiratory activity.

18. The method of claim 15 delivering electrical stimulation includes delivering electrical stimulation that is in phase with the patient's expiratory activity.

19. The method of claim 15 delivering electrical stimulation includes delivering electrical stimulation that is in phase with the patient's inspiratory and expiratory activity.

20. The method of claim 15 wherein receiving an input signal and delivering electrical stimulation are performed by instructions contained by a computer-readable medium.

21. A method treating a patient, comprising:
implanting an electrical stimulator in a subclavicular region of the patient;
implanting four bilateral parasternal electrodes at the patient's second and third intercostal spaces, and connecting the electrodes to the electrical stimulator;
implanting one or more strain gages at the patient's chest wall and connecting the one or more strain gages to the electrical stimulator; and
at least reducing the patient's sensation of dyspnea by electrically stimulating the patient's intercostal muscles via the electrodes in response to sensing inspiration.

22. A system for treating a patient, comprising:
an electrical signal delivery electrode;
a feedback sensor positionable to sense a patient's breathing;
a controller coupled to the delivery electrode and the feedback sensor, the controller being programmed with instructions, that when executed:
receive an indication of the patient's breathing from the feedback sensor; and
at least reduce the patient's sensation of by directing to the signal delivery electrode an electrical signal that is in phase with the patient's breathing, based at least in part on the indication of the patient's breathing received from the feedback sensor.

23. The system of claim 22 wherein the signal delivery electrode is a skin-mounted electrode.

24. The system of claim 22 wherein the signal delivery electrode is an implantable electrode.

25. The system of claim 22 wherein the feedback sensor includes a strain gage.

26. The system of claim 22 wherein the feedback sensor includes a thermistor.

27. The system of claim 22 wherein the controller is an implantable controller.

28. The system of claim 22 wherein the controller is programmed with instructions that when executed direct an electrical signal that is in phase with the patient's inspiratory breathing activity.

29. The system of claim 22 wherein the controller is programmed with instructions that when executed direct an electrical signal that is in phase with the patient's expiratory breathing activity.

30. The system of claim 22 wherein the controller is programmed with instructions that when executed direct an electrical signal that is in phase with the patient's inspiratory breathing activity and the patient's expiratory breathing activity.
